(19) United States
(12) Patent Application Publication
(10) Pub. No.: US 2023/0201215 A1
(43) Pub. Date: Jun. 29, 2023

(54) DRUG COMBINATIONS FOR INHIBITING INFECTIVITY OF INFLUENZA AND CORONA VIRUSES

(71) Applicant: SystaMedic Inc., Clinton, CT (US)

(72) Inventor: Anton Franz Joseph FLIRI, Mystic, CT (US)

(73) Assignee: SystaMedic Inc., Clinton, CT (US)

(21) Appl. No.: 17/921,532

(22) PCT Filed: Apr. 27, 2021
(86) PCT No.: PCT/US2021/029388

§ 371 Date: Oct. 26, 2022

Related U.S. Application Data
(60) Provisional application No. 63/017,289, filed on Apr. 29, 2020.

(51) Int. Cl. A61K 31/55; A61K 31/245; A61K 31/138; A61K 31/167

(52) U.S. Cl. CPC A61K 31/55; A61K 31/245; A61K 31/138; A61K 31/167

(57) ABSTRACT

Drug combinations, compositions including pharmaceutical compositions as well as methods of using and treating a viral infection, the drug combinations including (1) protease inhibitors Camostat, Nafamostat, (2) modulators or inhibitors of intracellular steroid hormone receptors signaling pathways (steroid hormone receptor antagonists), (3) modulators or inhibitors of SKP2 protein, (4) modulators of poly(ADP-ribose) polymerases and (5) angiotensin 2 response modifiers.

### CROSS-REFERENCE TO RELATED APPLICATIONS

This application claims priority to and the benefit of U.S. Provisional Patent Application Ser. No. 63/017,289 filed Apr. 29, 2020, the disclosure of which is incorporated herein by reference in its entirety.

### FIELD

This disclosure relates to pharmaceutical compositions, pharmaceutical combinations and methods of treatment of viral infections.

### BACKGROUND

In virus classification, influenza viruses are RNA viruses that make up four of the seven genera of the family Orthomyxoviridae. Serotypes causing infections include H1N1, which caused Spanish flu in 1918; H2N2, which caused Asian Flu in 1957; H3N2, which caused Hong Kong Flu in 1968; H5N1, which caused Bird Flu in 2004; H1N2, endemic in humans, pigs and birds, and H7N9, which has been rated as having the greatest pandemic potential among the influenza virus A subtypes. Influenza viruses A, B, C, and D are very similar in overall structure. The viral particles of all influenza viruses are made of a viral envelope containing the glycoproteins hemagglutinin and neuraminidase wrapped around a central core. The central core contains the viral RNA genome and other viral proteins that package and protect this RNA. The cleavage of the influenza virus hemagglutinin (HA) by host cell proteases such as for example TMPRSS2 is essential for viral infectivity.

### SUMMARY

In one embodiment, a pharmaceutical composition is provided. The pharmaceutical composition includes (1) a TMPRSS2 inhibitor including camostat, nafamostat or combinations thereof; (2) an intracellular steroid hormone response modifier including toremifen, tamoxifen, clomifene, raloxifene, fulvestrant, flutamide, nilutamide, bicalutamide, cyproterone acetate, enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, Andrographis paniculata extracts and preparations, or combinations thereof; and a pharmaceutically acceptable carrier.

In another embodiment, a method for treating and/or preventing viral infections in a mammal is provided. The method includes administering to the mammal in need of such treatment an effective amount of a pharmaceutical composition including (1) a TMPRSS2 inhibitor including camostat, nafamostat or combinations thereof; (2) an intracellular steroid hormone response modifier including toremifen, tamoxifen, clomifene, raloxifene, fulvestrant, flutamide, nilutamide, bicalutamide, cyproterone acetate, enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, Andrographis paniculata extracts and preparations, or combinations thereof; and a pharmaceutically acceptable carrier.

In another embodiment, a pharmaceutical composition is provided. The pharmaceutical composition includes a poly(ADP-ribose) polymerase inhibitor including rucaparib, fisetin, tricetin, quercetin, myricetin, gossypetin, delphinidin, gallic acid, decursin, decursinol angelate, or combinations thereof; a viral replication inhibitor including remdesivir, favipiravir, triazavirin, umifenovir, ASC09, azvudine, danoprevir, darunavir, lopinavir, ritonavir, lopinavir+ritonavir, remdesivir, baloxavir, marboxil, molnupiravir or combinations thereof; and a pharmaceutically acceptable carrier.

In another embodiment, a pharmaceutical composition is provided. The pharmaceutical composition includes a poly(ADP-ribose) polymerase inhibitor including rucaparib, fisetin, tricetin, quercetin, myricetin, gossypetin, delphinidin, gallic acid, decursin, decursinol angelate, or combinations thereof; a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; and a pharmaceutically acceptable carrier.

In another embodiment, a pharmaceutical composition is provided. The pharmaceutical composition includes a poly(ADP-ribose) polymerase inhibitor including rucaparib, fisetin, tricetin, quercetin, myricetin, gossypetin, delphinidin, gallic acid, decursin, decursinol angelate, or combinations thereof; an intracellular steroid hormone response modifier including toremifen, tamoxifen, clomifene, raloxifene, fulvestrant, flutamide, nilutamide, bicalutamide, cyproterone acetate, Enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, or combinations thereof; and a pharmaceutically acceptable carrier.

In another embodiment, a pharmaceutical composition is provided. The pharmaceutical composition includes a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; a poly(ADP-ribose) polymerase inhibitor including rucaparib, fisetin, tricetin, quercetin, myricetin, gossypetin, delphinidin, gallic acid, decursin, decursinol angelate, or combinations thereof; and a pharmaceutically acceptable carrier.

In another embodiment, a method for treating and/or preventing viral infections in a mammal is provided. The method includes administering to the mammal in need of such treatment an effective amount of a pharmaceutical composition including a poly(ADP-ribose) polymerase inhibitor including rucaparib, fisetin, tricetin, quercetin, myricetin, gossypetin, delphinidin, gallic acid, decursin, decursinol angelate, or combinations thereof; a viral replication inhibitor including remdesivir, favipiravir, triazavirin, umifenovir, ASC09, azvudine, danoprevir, darunavir, lopinavir, ritonavir, lopinavir+ritonavir, remdesivir, baloxavir marboxil, molnupiravir or combinations thereof; and a pharmaceutically acceptable carrier.

In another embodiment, a method for treating and/or preventing viral infections in a mammal is provided. The method includes administering to the mammal in need of such treatment an effective amount of a pharmaceutical composition including a poly(ADP-ribose) polymerase inhibitor including rucaparib, fisetin, tricetin, quercetin, myricetin, gossypetin, delphinidin, gallic acid, decursin, decursinol angelate, or combinations thereof; a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; and a pharmaceutically acceptable carrier.

In another embodiment, a method for treating and/or preventing viral infections in a mammal is provided. The method includes administering to the mammal in need of such treatment an effective amount of a pharmaceutical composition including a poly(ADP-ribose) polymerase inhibitor including rucaparib, fisetin, tricetin, quercetin, myricetin, gossypetin, delphinidin, gallic acid, decursin, decursinol angelate, or combinations thereof; an intracellular steroid hormone response modifier including toremifen, tamoxifen, clomifene, raloxifene, fulvestrant, flutamide, nilutamide, bicalutamide, cyproterone acetate, Enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, or combinations thereof; and a pharmaceutically acceptable carrier.

In another embodiment, a method for treating and/or preventing viral infections in a mammal is provided. The method includes administering to the mammal in need of such treatment an effective amount of a pharmaceutical composition including a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; a poly(ADP-ribose) polymerase inhibitor including rucaparib, fisetin, tricetin, quercetin, myricetin, gossypetin, delphinidin, gallic acid, decursin, decursinol angelate, or combinations thereof; and a pharmaceutically acceptable carrier.

### DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustration of the Cov-19 life cycle; and

FIG. 2 is a graphic illustration of the protein-protein interactions and cellular processes affected by members in this cluster regulating the expression of viral entry accelerator TMPRSS2 and the viral attachment protein ACE.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

Afflicting a large segment of an unprepared population, infections caused by influenza and coronavirus including SAR-CoV-2 threaten economies and millions of lives world-wide. For mitigating damages caused by these pathogens identification of effective treatments for reducing viral infectivity and severity of infections are particularly useful. Towards this end identification of drugs with different molecular mechanisms of actions (MOA) capable of producing synergistic antiviral effects reduce likelihood of resistance development and are therefore particularly useful.

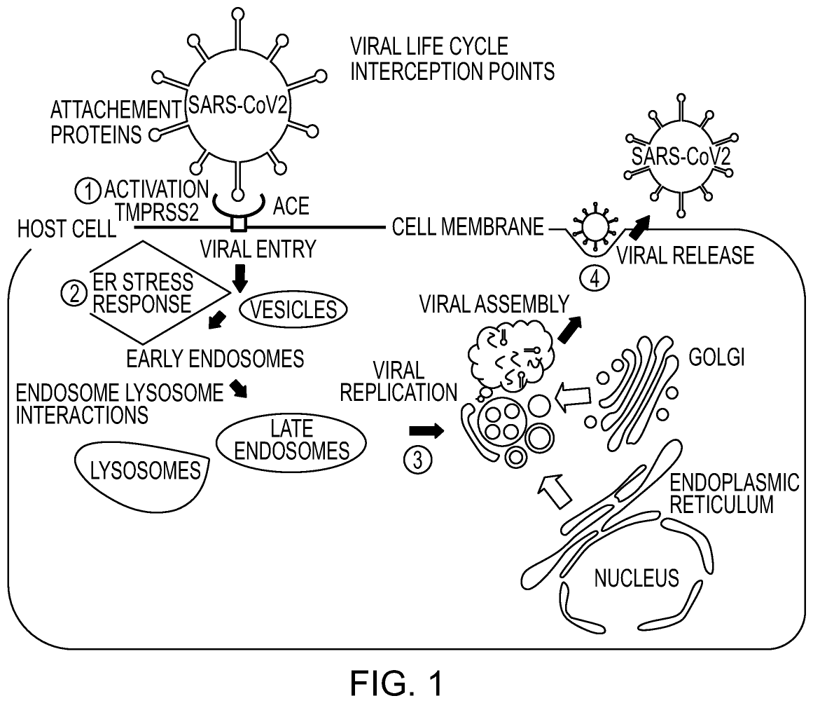

Coronaviruses (CoVs, in turn, are enveloped viruses with a positive-sense, single-stranded RNA genome, with subtypes alpha, beta and gamma. HCoV-229E or HCoV-OC43 belong to the alpha subtype and cause the common cold whereas Human coronavirus CoV-19, Human coronavirus HKU1, Severe Acute Respiratory Syndrome coronavirus (SARS-CoV-2) and Middle Eastern respiratory syndrome coronavirus (MERS-CoV) cause severe respiratory illnesses. The makeup of the viral genome, functions of viral proteins and mechanisms involved in viral replication cycles and the ability of these viruses to evade cellular defense mechanisms and host immune response are understood. (Schoeman, D., Fielding, B. C. Coronavirus envelope protein: current knowledge. Virol J 16, 69 (2019). https://doi.org/10.1186/s12985-019-1182-0; Shi, Y., Wang, Y., Shao, C. et al. COVID-19 infection: the perspectives on immune responses. Cell Death Differ (2020). https://doi.org/10.1038/s41418-020-0530-3, which are hereby incorporated by reference in their entireties) Like Influenza viruses, SARS-CoV-2 infectivity that is causing the current Cov-19 pandemic is aided by the serine protease TMPRSS2 (1) in FIG. 1. Like many other viruses, SARS-CoV-2 lacks the necessary machinery for self-replication and hijacks host protein-protein interactions (PPI) for evading host defenses and to complete viral life cycles as shown in section 3 of FIG. 1 which illustrates components of the SARS CoV-2 life cycle and host protein interactions involved. (Shi, Yaling, et al. “Immunopathological characteristics of coronavirus disease 2019 cases in Guangzhou, China.” medRxiv (2020).; Gassen, N.C., Niemeyer, D., Muth, D. et al. SKP2 attenuates autophagy through Beclin1-ubiquitination and its inhibition reduces MERS-Coronavirus infection. Nat Commun 10, 5770 (2019), which are hereby incorporated by reference in their entireties)

FIG. 1. Key steps in the Cov-19 life cycle. FIG. 1 shows cellular processes involved in SARS-CoV-2 cell entry and interactions of viral envelope proteins with host proteins (1) in FIG. 1. These interactions trigger cellular stress induced defense responses shown as (2) in FIG. 1. Viral evasion of cellular defense responses responses leads to viral replication (3) and shedding of new viral particles (4).

Network representations of key protein interactions involved in regulating cellular processes involved in the viral replication cycle shown in FIG. 1 can be created using protein network nodes identified in Table 1 shown below using the STRING Platform. (Szklarczyk D et al. STRING v11: protein-protein association networks with increased coverage, supporting functional discovery in genome-wide experimental datasets. Nucleic Acids Res. 2019 January; 47:D607-613), which is hereby incorporated by reference in its entirety) Targeting these virus-host protein interactions is anticipated to reduce SARS-COV-2 infections and severity of illness.

TABLE 1
Protein Network nodes for constructing protein interaction
networks regulating cellular processes illustrated
in FIG. 1 using the String platform.

| | | | |
|---|---|---|---|
| ACE2 | CTSB | KIT | SQSTM1 |
| ACR | CXCL10 | LAMP1 | STAT1 |
| APP | DAPK | LAMP2 | STAT3 |
| AR | DDX58 | LARGE | TANK |
| ATF4 | DES | MARCO | TAT |
| ATF6 | DPP4 | MAX | TBK1 |
| ATP1A1 | EGF | MET | TF |
| BCL2L1 | FBL1 | MPP5 | TFEB |
| BND7 | FGF2 | MTOR | TG |
| C2 | GADD153 | NPS | TLR3 |
| CAMP | GSK3 | PALM | TLR4 |
| CASP1 | IFNAR1 | PERP | TMPRSS2 |
| CAST | IL10 | REST | TNF |
| CD86 | IL1B | SDCBP | TNFRSF10B |
| CHOP | IL6 | SERCA2B | TP53 |
| CLOCK | IRF3 | SIRT1 | TP63 |
| CP | ISG15 | SKP2 | TYR |
| CS | KIR2DL1 | SON | UCHL1 |

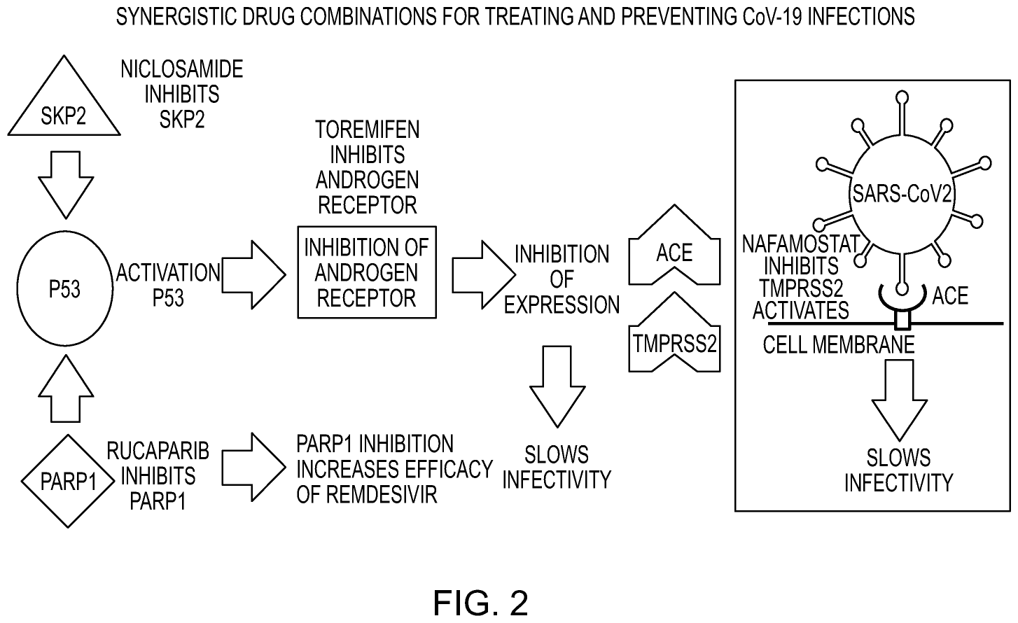

For pinpointing viral infection interception points, virus host protein interactions with SARSCoV-2 was analyzed using a methodology described in U.S. Pat. No. 10,242,091 and international application PCT/US2016/063792, which are hereby incorporated by reference in their entireties. For this purpose, protein-protein interaction networks regulating cellular processes shown in FIG. 1 by modulating interactions between proteins identified in Table 1 (key host proteins affected by interactions with SARS-CoV-2) are sub divided into 1054 smaller protein network fragments (called subnetworks) using gene enrichment analysis. These 1054 overlapping structure-function constraint protein subnetworks were used in a high-volume data extraction step for identifying information density measurements associated with viruses and drugs in each of the 1054 subnetworks as described in U.S. Pat. No. 10,242,091 and international application PCT/US2016/063792. Hierarchical clustering of the 1054 information density measurements associated with viruses and drugs identifies groups of viruses and drugs (clusters) that induce similar crosslinking between 1054 overlapping structure-function constraint protein subnetworks which is indicative of the induction of similar protein-protein interactions and information transfer between the 1054 overlapping structure-function constraint protein subnetworks and hence identifies functional similarities between viruses and drugs. Among members of a cluster containing the SARS CoV-2 coronavirus are Poly [ADP-ribose] polymerase inhibitor Rucaparib, protease inhibitors Camostat, Nafamostat, and estrogen modulators such as toremifene and Cupressus sempervirens extracts. (Siddiqui, Jawed A., et al. “8, 8”-Biapigeninyl stimulates osteoblast functions and inhibits osteoclast and adipocyte functions: Osteoprotective action of 8, 8″-biapigeninyl in ovariectomized mice.” Molecular and cellular endocrinology 323.2 (2010): 256-267, which is hereby incorporated by reference in its entirety)). FIG. 2 illustrates the functional similarities between members of the SARS CoV-2 coronavirus containing cluster in that cluster members affect interactions between proteins shown in Table 2 which regulate cellular processes that affect the expression of viral entry accelerator protein TMPRSS2 and the viral attachment protein ACE via modulation of the activity of intracellular steroid hormone receptors such as the androgen receptor via activating the cellular tumor antigen p53 by inhibiting S-phase kinase-associated protein 2 SKP2, and poly(ADP-ribose) polymerase PARP1 family members.

TABLE 2
Proteins involved in the regulation of cellular processes
that affect the expression of viral entry accelerator
protein TMPRSS2 and the viral attachment protein ACE.

| | |
|---|---|
| ACE2 | ISG15 |
| PARP1 | PERP |
| AR | SIRT1 |
| C2 | SKP2 |
| CARM1 | SQSTM1 |
| CASP1 | TANK |
| CLOCK | TMPRSS2 |
| CTSB | TP53 |
| DDIT3 | TP63 |
| DDX58 | UCHL1 |
| DPP4 | USP14 |

Thus, protease inhibitors Camostat and Nafamostat identified in our analysis are known to inhibit the proteolytic activity of TMPRSS2. The Estrogen response modifier toremifene or androgen receptor antagonists in turn, are predicted to down-regulate the expression levels of protease TMPRSS2 (Setlur S R, at al., (2008) Estrogen-dependent signaling in a molecularly distinct subclass of aggressive prostate cancer. J Natl Cancer Inst. 2008 Jun. 4; 100(11):815-25), which is hereby incorporated by reference in its entirety)), and virus attachment protein ACE2 thus, identifying that a combination of Camostat and Toremifene is expected to produce synergistic antiviral effects. (Douglas R. Green; Ghostly metabolic messages from dying cells. Nature 580, 36-37 (2020), which is hereby incorporated by reference in its entirety). Likewise, the inhibition of the SKP2 protein by niclosamide is expected to upregulate P53 activity which, in turn, is expected to down-regulate androgen receptor activity expected to cause concomitant decreases of ACE2, TMPRS2 expression levels which are known to be involved in accelerating the cellular uptake and hence increasing the infectivity of SARS CoV-2. Accordingly, combinations of Camostat and/or Toremifen with SKP2 inhibitors are expected to reduce cellular uptake of SARS CoV 2 and to produce synergistic anti-infective activities. Likewise, the inhibition of poly(ADP-ribose) polymerases PARP1 by Rucaparib leads to the upregulation of P53 activity which, in turn, down-regulates androgen receptor activity and ACE2, TMPRS2 expression levels. Thus, combinations of Camostat and/or Toremifen with inhibitors of poly(ADP-ribose) polymerases such as for example, rucaparib, are expected to decrease cellular uptake of SARS CoV-2, to lower infectivity rates which is expected to make hosts more resistant to viral infection. Moreover, inhibitors of poly(ADP-ribose) polymerases such as for example, rucaparib, are expected to affect the efficacy of viral replication (Brady, Pamlea N., et al. “Poly (ADP-ribose) polymerases in host-pathogen interactions, inflammation, and immunity.” Microbiology and Molecular Biology Reviews 83.1 (2019) and hence to synergize with inhibitors of RNA-dependent RNA polymerase such as for example Remdesivir. Accordingly, combinations inhibitors of RNA-dependent RNA polymerase such as for example Remdesivir administered at amounts between 10 and 200 mg in combination with a poly(ADP-ribose) polymerases inhibitors such as for example rucaparib administered at amounts between 100 and 600 mg are useful for treatment of viral infections.

Aspects and embodiments of the present disclosure include drug combinations, compositions including pharmaceutical compositions as well as methods of using to treat and/or prevent viral infections, the drug combinations including (1) protease inhibitors, including TMRPSS2 protease inhibitors, such as, for example, Camostat administered in amounts between about 100 mg to about 900 mg, Nafamostat administered in amounts between about 10 mg to about 50 mg, or a combination thereof to decrease influenza and coronavirus infectivity by inhibiting for example, protease TMRPSS2 which is an enzyme that accelerates viral entry into cells, and (2) modulators or inhibitors of intracellular steroid hormone receptors signaling pathways (steroid hormone receptor antagonists) such as, for example, toremifen administered in amounts between about 60 mg to about 200 mg, tamoxifen administered in amounts between about 10 mg to about 20 mg, clomifene administered in amounts between about 50 mg to about 100 mg, raloxifene administered in amounts between about 30 mg to about 150 mg, fulvestrant administered in amounts between about 5 mg to about 20 mg, flutamide administered in amounts between about 50 mg to about 250 mg, filutamide, bicalutamide, cyproterone acetate administered in amounts between about 5 mg and about 100 mg, enzalutamide, diethylstilbestrol, hydroxyflutamide, apalutamide, darolutamide, enzalutamide administered in amounts between 5 mg to about 100 mg, proxalutamide, cimetidine, topilutamide, alfatradiol, dutasteride, epristeride, finasteride, chlormadinone acetate, megestrol acetate, osaterone acetate, nomegestrol acetate, atraric acid administered in amounts between about 100 mg to about 300 mg, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, Andrographis paniculata extracts and preparations or combinations thereof to decrease steroid hormone receptor activity-mediated protein expression levels of TMPRSS2.

For enhancing the compositions, combinations and methods, embodiments of the present disclosure can also include in addition one or more of an inhibitor of S-phase kinase-associated protein 2 (SKP2), such as, for example niclosamide administered in amounts between about 500 mg to about 1500 mg, valinomycin administered in sub toxic amounts, nobiletin administered in amounts between about 1 mg to about 15 mg, dioscin administered in amounts between about 1 mg to about 50 mg, paeoniflorin administered in amounts between about 30 mg to about 60 mg, cafestol administered in amounts between about 50 mg to about 1000 mg, paeony root extracts, citrus peel extracts, Dioscorea extracts, Platycodon extracts, Melilotus extracts, Diospyros chinensis extracts, Coreopsis tinctoria extracts or combinations thereof, can be particularly useful. Moreover, the embodiments of the present disclosure can also include in addition inhibitors of poly(ADP-ribose) polymerases PARP 1-12, such as, for example, rucaparib administered at amounts between 100 and 600 mg, Olaparib administered at amounts between about 50 mg to about 200 mg, niraparib administered at amounts between about 200 mg to about 600 mg, talazoparib administered at amounts between about 10 mg to about 60 mg, veliparib administered at amounts between about 10 mg to about 60 mg, pamiparib administered at amounts between about 60 mg to about 120 mg, CEP 9722, E7016, fisetin, tricetin, quercetin, myricetin, gossypetin, delphinidin, gallic acid, decursin, decursinol angelate, or combinations thereof which are especially useful for deceasing infectivity of influenza and coronaviruses and can also include the use of these drug combinations either alone or in combination with antivirals, such as, for example, immunoglobulin, interferon alpha, interferon gamma, interleukin-2, favipiravir, triazavirin, umifenovir, ASC09, azvudine, danoprevir, darunavir, lopinavir, ritonavir, lopinavir+ritonavir, remdesivir, baloxavir marboxil, chloroquine and hydroxychloroquine, dihydroartemisinin, carriomycin, Suramin sodium, molnupiravir administered in amounts between about 100 mg to about 800 mg, corticosteroids, fingolimod, leflunomide, thalidomide, kinase inhibitors, jakotinib hydrochloride, ruxolitinib, adalimumab, camrelizumab, eculizumab, mepolizumab, PD-1 mAb, tocilizumab, acetylcysteine, angiotensin receptor blockers, angiotensin converting enzyme (ACE) inhibitors, bismuth potassium citrate, bromhexine hydrochloride, diammonium glycyrrhizinate, dipyridamole, ebastine, hydrogen peroxide, inhaled gases, pirfenidone, polyinosinic-, polycytidylic acid, rhG-CSF, thymosin, tranilast, vitamin C, adamumab+tozumab, or combinations thereof for treating for treating viral infections including, for example, influenza and coronavirus infections.

The embodiments of the present disclosure can also include in addition antivirals, such as, for example, immunoglobulin, interferon alpha, interferon gamma, interleukin-2, favipiravir, triazavirin, umifenovir, ASC09, azvudine, danoprevir, darunavir, lopinavir, ritonavir, lopinavir+ritonavir, remdesivir, baloxavir marboxil, molnupiravir, chloroquine and hydroxychloroquine, dihydroartemisinin, carriomycin, Suramin sodium, corticosteroids, fingolimod, leflunomide, thalidomide, kinase inhibitors, jakotinib hydrochloride, ruxolitinib, adalimumab, camrelizumab, eculizumab, mepolizumab, PD-1 mAb, tocilizumab, acetylcysteine, angiotensin receptor blockers, angiotensin converting enzyme (ACE) inhibitors, bismuth potassium citrate, bromhexine hydrochloride, diammonium glycyrrhizinate, dipyridamole, ebastine, hydrogen peroxide, inhaled gases, pirfenidone, polyinosinic-, polycytidylic acid, rhG-CSF, thymosin, tranilast, vitamin C, adamumab+tozumab, or combinations thereof for treating viral infections including, for example, influenza and coronavirus infections.

Preferred treatment or prevention of viral infections using the drug combinations, compositions including pharmaceutical compositions as well as methods of using to treat and/or prevent viral infections included in the present disclosure include the administration of amounts of said compositions between about 400 mg to about 2,000 mg by mouth depending on body weight twice to four times per day for 7-21 consecutive days. It is generally known to someone skilled in the art that said amounts of pharmaceutical compositions can also be administered via nasogastric, percutaneous endoscopic gastrostomy or orogastric tube and, if desired, said administration may include nasogastric or orogastric feeding.

Embodiments of the present disclosure also include the further addition of angiotensin 2 response modifiers including retinoic acid gamma receptor antagonists, renin inhibitors, angiotensin 1 receptor antagonists, ACE2 activators, including aliskiren, azilsartan, benazepril, candesartan, candesartan, cilexetil, captopril, cilnidipine, diminazene, enalapril, enalaprilat, equol, fosinopril, imidapril, irbesartan, liriope, lisinopril, losartan, magnolol, olmesartan, oltipraz, perindopril, perindoprilat, R667, ramipril, spironolactone, telmisartan, trigonelline, valsartan, or combinations thereof.

Embodiments of the present disclosure include drug combinations, compositions including pharmaceutical compositions as well as methods of using to treat and/or prevent viral infections, the drug combinations including a poly(ADP-ribose) polymerase inhibitor including rucaparib, fisetin, tricetin, quercetin, myricetin, gossypetin, delphinidin, gallic acid, decursin, decursinol angelate, or combinations thereof in combination with a viral replication inhibitor including remdesivir, molnupiravir, favipiravir, triazavirin, umifenovir, ASC09, azvudine, danoprevir, darunavir, lopinavir, ritonavir, lopinavir+ritonavir, remdesivir, molnupiravir, baloxavir marboxil, or combinations thereof. Amounts of compounds used herein can be the same as included in other embodiments of the present disclosure.

Embodiments of the present disclosure include drug combinations, compositions including pharmaceutical compositions as well as methods of using to treat and/or prevent viral infections, the drug combinations including a poly(ADP-ribose) polymerase inhibitor including rucaparib, fisetin, tricetin, quercetin, myricetin, gossypetin, delphinidin, gallic acid, decursin, decursinol angelate, or combinations thereof in combination with a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof. Amounts of compounds used herein can be the same as included in other embodiments of the present disclosure.

Embodiments of the present disclosure include drug combinations, compositions including pharmaceutical compositions as well as methods of using to treat and/or prevent viral infections, the drug combinations including a poly(ADP-ribose) polymerase inhibitor including rucaparib, fisetin, tricetin, quercetin, myricetin, gossypetin, delphinidin, gallic acid, decursin, decursinol angelate, or combinations thereof in combination with an intracellular steroid hormone response modifier including toremifen, tamoxifen, clomifene, raloxifene, fulvestrant, flutamide, nilutamide, bicalutamide, cyproterone acetate, Enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, or combinations thereof. Amounts of compounds used herein can be the same as included in other embodiments of the present disclosure.

Embodiments of the present disclosure include drug combinations, compositions including pharmaceutical compositions as well as methods of using to treat and/or prevent viral infections, the drug combinations including a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof in combination with a poly(ADP-ribose) polymerase inhibitor including rucaparib, fisetin, tricetin, quercetin, myricetin, gossypetin, delphinidin, gallic acid, decursin, decursinol angelate, or combinations thereof. Amounts of compounds used herein can be the same as included in other embodiments of the present disclosure.

In addition, the pharmaceutical compositions, combinations and method of treatment embodiments of the present disclosure can be used not only for humans but also for other mammals as well including animals.

Examples of animals include mammals, birds, reptiles, amphibians, and fish.

Embodiments of the present disclosure also include methods of treating and/or preventing viral infections, such as, for example, administering to a mammal in need of such treatment an effective amount of one or more of the pharmaceutical compositions or combinations embodiments of the present disclosure.

The embodiments of the present disclosure include pharmaceutical compositions and combinations of the present disclosure that are useful for treatment and/or prevention and methods of treatment and/or prevention of viral infections in a mammal including viruses such as, for example, influenza viruses, such as, for example, influenza viruses A, B, C, and D and coronaviruses, such as, for example, HCoV-229E, HCoV-OC43, Human coronavirus CoV-19, Human coronavirus HKU1, SARS-CoV-2 and MERS-CoV.

A pharmaceutical composition and embodiments thereof of the present disclosure can be directly administered as the preventing or ameliorating agent for viral infections of the present disclosure. However, it is desirable to provide the agent in the form of a variety of drug formulations. Such drug formulation contains combinations of active ingredients. Further, the drug formulation can be produced by an arbitrary method that has been well known in the art of drug formulation by mixing the active ingredient with at least one type of pharmacologically acceptable carrier or vehicle. It is desirable that the most effective administration route of drug formulation would be selected for treatment. Examples thereof include oral administration and parenteral administration such as intravenous, intraperitoneal, or subcutaneous administration. However, oral administration is preferable. Examples of dosage forms that can be used for administration include: oral agents such as tablets, powders, granules, pills, suspensions, emulsions, infusions and decoctions, capsules, syrups, liquid, elixirs, extracts, tinctures, and fluid extracts; and parenteral agents such as parenteral injections, intravenous fluids, creams, and suppositories while suitable inhalable formulations for pulmonary or nasal administration include aerosolized or nebulized preparations, such as liquids and powders, including dry powder, administered using an acceptable inhalation delivery device, such as, for example, a nebulizer. Such inhalable formulations can include a powdered composition that contains finely dispersed solid particles that are capable of (i) being readily dispersed in an inhalation device and (ii) inhaled by a subject so that a portion of the particles reach the lungs to permit penetration into the alveoli. Such a powder is considered to be “respirable” or suitable for pulmonary delivery. A composition suitable for “oral pulmonary administration” comprises particles at least a portion of which, when delivered via inhalation by the mouth, reach the tissues of the lung. The composition in the form of an oral composition is preferably used.

In the case involving the use of liquid preparations such as syrup appropriate for oral administration, the preparations can be formulated by addition of water, Sugars such as Sucrose, Sorbitol, and fructose; glycols such as polyethylene glycol and propylene glycol, oils such as Sesame oil, olive oil, and soybean oil; antiseptics such as p-hydroxy benzoate esters; parahydroxy benzoate derivatives such as methyl parahydroxy benzoate; preservatives such as Sodium benzoate; and flavors such as Strawberry flavor and peppermint flavor.

In addition, in the case involving the use of tablets, powders, and granules that are appropriate for oral administration the preparations can be formulated by addition of sugar such as lactose, glucose, sucrose, mannitol, and sorbitol; starch from potatoes, wheat, and corn; an inorganic substance such as calcium carbonate, calcium sulfate, sodium bicarbonate, and sodium chloride; an excipient of a plant-derived powder such as crystalline cellulose, a sweetroot powder and gentian powder, a disintegrator Such as starch, agar, gelatin powder, crystalline cellulose, carmellose sodium, carmellose calcium, calcium carbonate, sodium bicarbonate, and sodium alginate; a lubricant such as magnesium stearate, talc, hydrogenated plant oil, macrogol, and silicone oil; a binder such as polyvinyl alcohol, hydroxypropyl cellulose, methyl cellulose, ethylcellulose, carmellose, gelatin, and starch paste liquid; a surfactant such as fatty acid ester, and a plasticizer such as glycerine.

It is also possible to add an additive generally used for foods and beverages to the drug formulation appropriate for oral administration. Examples of additives include sweeteners, colorants, preservatives, thickening stabilizers, antioxidants, coloring agents, bleaches, antifungal agents, gumbases, bittering agents, enzymes, gloss agents, acidulants, seasonings, emulsifiers, fortifiers, production agents, aroma chemicals, and spice extracts.

The pharmaceutical composition formulation embodiments of the present disclosure appropriate for oral administration may be directly used in the form of, for example, a powder food product, a sheet-type food product, a bottled food product, a canned food product, a retort food product, a capsule food product, a tablet food product, a liquid food product, or a drink. In addition, the drug formulation may be used in the form of food or beverage such as health food, functional food, nutritional supplement, or food for specified health use for prevention and/or treatment of viral infections. For example, a parenteral injection appropriate for parenteral administration comprises preferably a sterilized aqueous agent which contains a composition of the present disclosure and which is isotonic to the blood of a recipient. For example, for a parenteral injection, an injectable solution is prepared with the use of a pharmaceutically acceptable carrier or vehicle comprising a salt solution, a glucose solution, or a mixture of a salt Solution and a glucose solution.

In addition, it is also possible to add at least one supplemental component to a parenteral agent, wherein Such components can be selected from the group consisting of diluents, antiseptics, flavors, excipients, disintegrators, lubricants, binders, Surfactants, and plasticizers, which are described above for an oral agent.

All publications, including but not limited to, issued patents, patent applications, and journal articles, cited in this application are each herein incorporated by reference in their entirety.

Thus, while there have been shown, described and pointed out, fundamental novel features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that compositions and/or structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A pharmaceutical composition comprising (1) a TMPRSS2 inhibitor including camostat, nafamostat or combinations thereof; (2) an intracellular steroid hormone response modifier including toremifen, tamoxifen, clomifene, raloxifene, fulvestrant, flutamide, nilutamide, bicalutamide, cyproterone acetate, enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, Andrographis paniculata extracts and preparations, or combinations thereof; and a pharmaceutically acceptable carrier.

2. The pharmaceutical composition according to claim 1, further including an SKP2 inhibitor comprising niclosamide, valinomycin, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts or combinations thereof.

3. The pharmaceutical composition according to claim 1, further including an inhibitor of poly(ADP-ribose) polymerases comprising rucaparib, olaparib, niraparib, talazoparib, veliparib, pamiparib, CEP 9722, E7016, fisetin, tricetin, quercetin, myricetin, gossypetin, delphinidin, gallic acid, decursin, decursinol angelate or combinations thereof.

4. The pharmaceutical composition according to claim 1 wherein the TMPRSS2 inhibitor is Nafamostat

5. The pharmaceutical composition according to claim 1, wherein the TMPRSS2 inhibitor is Camostat

6. The pharmaceutical composition according to claim 1, wherein the intracellular steroid hormone response modifier is Toremifen

7. The pharmaceutical composition according to claim 2, wherein the SKP2 inhibitor is niclosamide

8. The pharmaceutical composition according to claim 3 wherein the inhibitor of poly(ADP-ribose) polymerases is Rucaparib

9. The pharmaceutical composition according to claim 1, further comprising an antiviral including immunoglobulin, interferon alpha, interferon gamma, interleukin-2, favipiravir, triazavirin, umifenovir, ASC09, azvudine, danoprevir, darunavir, lopinavir, ritonavir, lopinavir+ritonavir, remdesivir, molnupiravir, baloxavir marboxil, chloroquine and hydroxychloroquine, dihydroartemisinin, carriomycin, Suramin sodium, corticosteroids, fingolimod, leflunomide, thalidomide, kinase inhibitors, jakotinib hydrochloride, ruxolitinib, adalimumab, camrelizumab, eculizumab, mepolizumab, PD-1 mAb, tocilizumab, acetylcysteine, angiotensin receptor blockers, angiotensin converting enzyme (ACE) inhibitors, bismuth potassium citrate, bromhexine hydrochloride, diammonium glycyrrhizinate, dipyridamole, ebastine, hydrogen peroxide, inhaled gases, pirfenidone, polyinosinic-, polycytidylic acid, rhG-CSF, thymosin, tranilast, vitamin C, adamumab+tozumab, or combinations thereof.

10. The pharmaceutical composition according to claim 1, including wherein the steroid hormone response modifier include atraric acid, zinc salts, diindolylmethane, melatonin, Paeonia lactiflora extracts and preparations, Glycyrrhiza glabra extracts and preparations, Ganoderma lucidum extracts and preparations, or combinations thereof.

11. The pharmaceutical composition according to claim 2, further comprising a SKP2 inhibitor including nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof.

12. The pharmaceutical composition according to claim 3, further comprising a poly(ADP-ribose) polymerase inhibitor including fisetin, tricetin, quercetin, myricetin, gossypetin, delphinidin, gallic acid, decursin, decursinol angelate, or combinations thereof.

13. The pharmaceutical composition according to claim 1, further comprising an angiotensin2 response modifier including aliskiren, azilsartan, benazepril, candesartan, candesartan, cilexetil, captopril, cilnidipine, diminazene, enalapril, enalaprilat, equol, fosinopril, imidapril, irbesartan, liriope, lisinopril, losartan, magnolol, olmesartan, oltipraz, perindopril, perindoprilat, R667, ramipril, spironolactone, telmisartan, trigonelline, valsartan, or combinations thereof.

14. The pharmaceutical composition according to claim 2, further comprising an antiviral agent including immunoglobulin, interferon alpha, interferon gamma, interleukin-2, favipiravir, triazavirin, umifenovir, ASC09, azvudine, danoprevir, darunavir, lopinavir, ritonavir, lopinavir+ritonavir, remdesivir, molnupiravir, baloxavir marboxil, chloroquine and hydroxychloroquine, dihydroartemisinin, carriomycin, Suramin sodium, corticosteroids, fingolimod, leflunomide, thalidomide, kinase inhibitors, jakotinib hydrochloride, ruxolitinib, adalimumab, camrelizumab, eculizumab, mepolizumab, PD-1 mAb, tocilizumab, acetylcysteine, angiotensin receptor blockers, angiotensin converting enzyme (ACE) inhibitors, bismuth potassium citrate, bromhexine hydrochloride, diammonium glycyrrhizinate, dipyridamole, ebastine, hydrogen peroxide, inhaled gases, pirfenidone, polyinosinic-, polycytidylic acid, rhG-CSF, thymosin, tranilast, vitamin C, adamumab+tozumab, or combinations thereof.

15. A pharmaceutical composition comprising a poly(ADP-ribose) polymerase inhibitor including rucaparib, fisetin, tricetin, quercetin, myricetin, gossypetin, delphinidin, gallic acid, decursin, decursinol angelate, or combinations thereof; a viral replication inhibitor including remdesivir, molnupiravir, favipiravir, triazavirin, umifenovir, ASC09, azvudine, danoprevir, darunavir, lopinavir, ritonavir, lopinavir+ritonavir, remdesivir, baloxavir marboxil, or combinations thereof; and a pharmaceutically acceptable carrier.

16. A pharmaceutical composition comprising a poly(ADP-ribose) polymerase inhibitor including rucaparib, fisetin, tricetin, quercetin, myricetin, gossypetin, delphinidin, gallic acid, decursin, decursinol angelate, or combinations thereof; a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; and a pharmaceutically acceptable carrier.

17. A pharmaceutical composition comprising a poly(ADP-ribose) polymerase inhibitor including rucaparib, fisetin, tricetin, quercetin, myricetin, gossypetin, delphinidin, gallic acid, decursin, decursinol angelate, or combinations thereof; an intracellular steroid hormone response modifier including toremifen, tamoxifen, clomifene, raloxifene, fulvestrant, flutamide, nilutamide, bicalutamide, cyproterone acetate, Enzalutamide, diethylstilbestrol, hydroxyflutamide, apalutamide, darolutamide, enzalutamide, proxalutamide, cimetidine, topilutamide, alfatradiol, dutasteride, epristeride, finasteride, chlormadinone acetate, megestrol acetate, osaterone acetate, nomegestrol acetate, atraric acid, zinc salts, diindolylmethane, melatonin, Paeonia lactiflora extracts and preparations, Glycyrrhiza glabra extracts and preparations, Ganoderma lucidum extracts and preparations, Cupressus sempervirens extracts and preparations, or combinations thereof; and a pharmaceutically acceptable carrier.

18. A pharmaceutical composition comprising a SKP2 inhibitor including niclosamide, nobiletin, dioscin, paeoniflorin, cafestol, paeony root extracts, citrus peel extracts, Dioscorea extracts, Platycodon extracts, Melilotus extracts, Diospyros chinensis extracts, Coreopsis tinctoria extracts, or combinations thereof; a poly(ADP-ribose) polymerase inhibitor including rucaparib, fisetin, tricetin, quercetin, myricetin, gossypetin, delphinidin, gallic acid, decursin, decursinol angelate, or combinations thereof; and a pharmaceutically acceptable carrier.

19. A method for treating and/or preventing viral infections in a mammal, the method comprising administering to said mammal in need of such treatment an effective amount of a pharmaceutical composition according to claim 1.

20. A method for treating and/or preventing viral infections in a mammal, the method comprising administering to said mammal in need of such treatment an effective amount of a pharmaceutical composition according to claim 2.
